(19) United States
(12) Patent Application Publication
(10) Pub. No.: US 2007/0248527 A1
(43) Pub. Date: Oct. 25, 2007

(54) METHODS AND SYSTEMS FOR SELECTIVELY SEPARATING CO2 FROM AN OXYGEN COMBUSTION GASEOUS STREAM

(76) Inventor: Dwain F. Spencer, Redding, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/380,117

(22) Filed: Apr. 25, 2006

(51) Int. Cl. B01D 53/62; C07C 9/00; B01D 53/14; C01B 31/20; B01J 19/00

(52) U.S. Cl. 423/437.1; 423/220; 422/198; 585/15

(57) ABSTRACT

Methods are provided for the selective multi-stage removal of CO₂ from an oxygen combustion flue gas stream to provide a CO₂ depleted gaseous stream. In practicing the subject methods, an initial flue gas stream is contacted with an aqueous fluid under conditions of CO₂ hydrate formation to produce a CO₂ hydrate slurry and CO₂ depleted gaseous stream. A feature of the subject methods is that the CO₂ hydrate slurry is separated from the CO₂ depleted gaseous stream and then compressed to high pressure to produce a high-pressure CO₂ product. A further feature is that the CO₂-depleted gaseous stream is sent to at least one additional hydrate reactor for further removal of CO₂. Also provided are systems that find use in practicing the subject methods. The subject methods and systems find use in a variety of applications where it is desired to remove CO₂ selectively from an oxygen combustion flue gas stream

### BACKGROUND

In many applications where mixtures of two or more gaseous components are present, it is often desirable to selectively remove one or more of the component gases from the gaseous stream. Of increasing interest in a variety of industrial applications, including power generation, chemical synthesis, natural gas upgrading, and conversion of methane hydrates to hydrogen and CO₂, is the selective removal of CO₂ from multi-component gaseous streams.

As man made CO₂ is increasingly viewed as a pollutant, an area in which it is desirable to separate CO₂ from a multi-component gaseous stream is in the area of pollution control. Emissions from industrial facilities, such as manufacturing and power generation facilities, often include CO₂. In such instances, it is often desirable at least to reduce the CO₂ concentration of the emissions. The CO₂ may be removed prior to combustion in some cases and post-combustion in others.

Various processes have been developed for removing or isolating a particular gaseous component from a multi-component gaseous stream. These processes include cryogenic fractionation, selective adsorption by solid adsorbents, gas absorption, and the like. In gas absorption processes, solute gases are separated from gaseous mixtures by transport into a liquid solvent. In such processes, the liquid solvent ideally offers specific or selective solubility for the solute gas or gases to be separated.

Gas absorption finds widespread use in the separation of CO₂ from multi-component gaseous streams. In CO₂ gas absorption processes that currently find use, the following steps are employed: (1) absorption of CO₂ from the gaseous stream by a host solvent, e.g., monoethanolamine; (2) removal of CO₂ from the host solvent, e.g., by steam stripping; and (3) compression of the stripped CO₂ for disposal, e.g., by sequestration through deposition in the deep ocean or in ground aquifers.

Although these processes have proved successful for the selective removal of CO₂ from a multi-component gaseous stream, they are energy intensive and expensive in terms of cost per ton of CO₂ removed or sequestered.

There is continued interest in the development of less expensive and/or energy intensive processes for the selective removal of CO₂ from multi-component gaseous streams. Of particular interest would be the development of an efficient process which could provide for efficient CO₂ separation from a flue gas stream that is rich in CO₂ and that contains primarily CO₂ and oxygen (O₂).

There is an increasing interest in this application as utilities and the federal government, primarily the Department of Energy (DOE), seek methods to reduce the performance and cost penalties associated with controlling emissions of CO₂ and other emissions, e.g. oxides of nitrogen, from power plants. The DOE is funding the development of new combustion processes that utilize high purity oxygen as the oxidant rather than air, which has a high nitrogen content, to combust natural gas, coal, and other carbon containing fuels.

By utilizing oxygen, the combustion products of flue gas are primarily water vapor (H₂O), CO₂, and excess O₂, which is necessary to assure complete combustion. The water vapor may be easily condensed, leaving a mixed gas stream, containing CO₂ and excess O₂. If the CO₂ is to be sequestered or utilized for secondary or tertiary oil recovery or methane gas recovery from deep coal bed seams, the CO₂ must be essentially free of O₂. Therefore, there is a need for the development of a cost effective, efficient process for separating the component gases of a mixed CO₂—O₂ gaseous stream, such as a CO₂—O₂ flue gas stream.

Relevant Literature

U.S. patents of interest include U.S. Pat. Nos. 5,700,311; 6,090,186; 6,106,595; 6,235,091; 6,235,092, 6,352,576, and 6,797,039.

### SUMMARY

Methods are provided for the selective removal of CO₂ from an oxidizing condition combustion stream, such as a flue gas stream containing primarily CO₂ and O₂, to provide a CO₂-depleted gaseous stream. Aspects of the methods include contacting the gaseous stream with an aqueous fluid under conditions of CO₂ hydrate formation to produce a CO₂ hydrate slurry and a CO₂-depleted gaseous stream. In certain embodiments, the CO₂ hydrate slurry is then pumped to high pressure and decomposed to produce a high-pressure CO₂ product gas stream. In certain embodiments, the CO₂-depleted gaseous stream is sent to at least one more hydrate reactor for further removal of CO₂. Also provided are systems that find use in practicing the subject methods. The subject methods and systems find use in a variety of applications where it is desired to remove CO₂ selectively from an oxygen combustion gaseous stream.

### BRIEF DESCRIPTION OF THE FIGURES

FIG. 1 provides a schematic diagram for separation of CO₂ from the flue gas stream from an oxygen-fired natural gas combined cycle power plant according to an embodiment of the subject invention.

### DETAILED DESCRIPTION

Methods are provided for the selective removal of CO₂ from a gaseous stream to provide a high-pressure CO₂ product gas stream and a CO₂-depleted gaseous stream. In practicing the subject methods, an initial gaseous stream is contacted with an aqueous fluid under conditions of selective CO₂ hydrate formation to produce a CO₂ hydrate slurry and CO₂-depleted gaseous stream, which are separated from each other. Aspects of the methods include embodiments where the CO₂ hydrate slurry is then pumped to high pressure and then decomposed to produce a high-pressure CO₂ product gas stream, e.g., for subsequent use or sequestration. Aspects of the invention further include embodiments where the CO₂-depleted gaseous stream is sent to at least one additional hydrate reactor for further removal of CO₂. The CO₂ concentration of the resultant CO₂—O₂ gas stream is low enough to permit venting the gas stream into the atmosphere or recycling of the oxygen rich stream back to the gas turbine combustor. Also provided are systems that find use in practicing the subject methods. The subject methods and systems find use in a variety of applications where it is desired to remove CO₂ selectively from a gaseous stream, such as a flue gas.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, representative embodiments of the subject methods are described first in greater detail, followed by a more detailed review of representative embodiments of systems of the subject invention.

Methods

As summarized above, the subject invention provides methods of removing CO₂ selectively from a gaseous stream containing primarily CO₂ and O₂. Embodiments of the subject method provide for resource efficient separation of the CO₂ from the gaseous stream at low pressures and produce a high-pressure CO₂ product from the separated CO₂. In certain embodiments of the invention, the high-pressure CO₂ product is a product having a pressure that is at least about 10 times higher, such as at least about 40-50 times higher, or even higher than the pressure at which the CO₂ separation from the gaseous stream occurred. Specific pressure ranges of interest for the CO₂ separation step and product of the subject process are provided below.

Because O₂ does not form hydrates under the thermodynamic, temperature, and pressure conditions necessary to form CO₂ hydrates, effective separation of the CO₂ from gaseous streams may be effected by CO₂ hydrate formation. The first step of the subject methods is to contact a gaseous stream with an aqueous fluid under conditions sufficient for CO₂ hydrate formation to occur.

In certain embodiments, the gaseous stream is a flue gas stream. In certain embodiments, the gaseous stream is a gas produced by combustion of an organic fuel (e.g., fuels containing primarily carbon) with high purity oxygen. Organic fuels of interest include, but are not limited to: natural gas (methane), bituminous coal, sub-bituminous coal, lignite, petroleum and petroleum residues, wood, carbon containing waste streams, and the like. By high purity oxygen is meant a gaseous stream that is at least 90% pure, such as at least 95% pure or purer oxygen. In certain embodiments, the gaseous stream that is subjected to the subject methods is a flue gas stream. The flue gas stream may be from high purity oxygen driven combustion of any of a variety of different fuels containing primarily carbon, e.g. natural gas, bituminous coal, sub-bituminous coal, lignite, petroleum and petroleum residues, wood, carbon containing waste streams, and the like.

The total pressure of the flue gas stream when contacted with the aqueous fluid in the first hydrate formation step of the subject methods may be as high as 10 atm or higher, and may range from about 15 to about 25 atm, such as from about 19 to about 21 atm and may be as high as 10 atm or higher.

The weight percentage of CO₂ in the flue gas streams amenable to treatment according to the subject invention may vary, and in certain embodiments ranges from about 85 to about 95%, such as from about 88 to about 94%, and including from about 89 to about 91%. The weight percentage of O₂ in the flue gas streams amenable to treatment according to the subject invention may range from about 5 to about 15%, such as from about 8 to about 12%, including from about 9 to about 11%. Small amounts of trace gases may also be present in the flue gas streams. If present, such traces gases are not present in amounts exceeding about 3 to 5%. The partial pressure of CO₂ in the flue gas stream (e.g., the stream from which water vapor has been previously condensed) need not be high, and may be as low as about 0.85 atm, including as low as about 0.83 atm.

In certain embodiments, the flue gas stream may have been preprocessed from its initial state prior to the first hydrate formation step of the subject methods. For example, in certain embodiments the pressure and/or temperature of the flue gas stream may have been modulated, e.g., raised or lowered, as desired and depending on the initial state of the flue gas stream. For example, flue gas may have a temperature of about 150° F. and a pressure of about 1 to 1.5 atm. The temperature of the gas may be lowered and/or the pressure of the gas may be raised to values desirable for hydrate formation. The initial gas source may also be split into one or more smaller streams, as desired.

In the first hydrate formation step of the present methods in which the flue gas is contacted with an aqueous fluid under CO₂ hydrate formation reaction conditions, any convenient aqueous fluid may be employed. Aqueous fluids of interest include, but are not limited to, water, either pure water or salt water, CO₂ nucleated water, e.g., as described in U.S. Pat. Nos. 5,700,311, 6,090,186, and 6,106,595, the disclosures of which are herein incorporated by reference, and the like.

In certain embodiments of interest, the aqueous fluid with which the flue gas stream is contacted may include a CO₂ hydrate promoter, as described in U.S. Pat. No. 6,352,576, the disclosure of which is herein incorporated by reference. In certain embodiments, the CO₂ hydrate promoters are low molecular weight proton donors, such as water-soluble halogenated hydrocarbons, and the like. Water soluble halogenated hydrocarbons of interest are generally those having from 1 to 5, such as 1 to 4, and including 3 to 4 carbon atoms, where the halogen moiety may be F, Cl, Br, or I. Specific halogenated hydrocarbons of interest include chloroform, ethylene chloride, carbon tetrachloride, and the like. Where the CO₂ hydrate promoter is ethylene chloride, it is generally dissolved in the nucleated water in an amount ranging from about 100 to 2500 ppm, such as from about 500 to 2000 ppm, and including from about 1000 to 1800 ppm. Where the CO₂ hydrate promoter is chloroform, it is generally present in the nucleated water in an amount ranging from about 100 to 2500 ppm, such as from about 500 to 2000 ppm, and including from about 1000 to 1800 ppm. Where the CO₂ hydrate promoter is carbon tetrachloride, it is generally present in the nucleated water in an amount ranging from about 50 to 200 ppm, such as from about 80 to 160 ppm, and including from about 100 to 120 ppm.

Also of particular interest as CO₂ hydrate promoters are organic salts, particularly low molecular weight alkyl ammonium, sulfonium, and phosphonium salts. The alkyl ammonium salts are compounds with cations of the generic formula:
R₄N⁺
Where each R may consist of linear or branched hydrocarbon elements of the formula:
CₐH₂ₐ₊₁
wherein a is an integer from 1 to 8. For example, R may be methyl or normal (linear) C₄H₉, but may also be iso-C₃H₇. The four groups attached to the nitrogen may be the same or different (i.e., one may be methyl while another may be ethyl, etc.). The anionic portion of the salt may consist of simple ions such as: F—, HCOO—, OH—, Br—, Cl—, NO₃—, etc, but may also be ions such as normal (linear): nCₐH₂ₐ₊₁ COO— or iso-nCₐH₂ₐ₊₁COO—.

The sulfonium salts may be compounds with cations of the generic formula:
R₃S⁺
where again R may be any of the possibilities cited above. Similarly, for the sulfonium salts, all three R groups need not be of the same chemical composition. The anion for the sulfonium salts may be F—. The phosphonium salts generally have the generic formula:
R₄P⁺
for the cations, with the same choices for the four R groups as described above. The anions may be anions as described above.

This class of alkyl-onium salts readily form hydrate structures involving encagement of the salt in Structure II or H class of polyhedral water cages. (In many cases the anion actually is part of the cage structure.) The hydrates of these salts form at or below atmospheric pressure and are stable well above the freezing point of water (where some melting points exceed 30° C.).

The above described “onium” salts vary widely in the number of water molecules per salt molecule (i.e., the hydration number). For example, the hydration number may be as low as 4 (for hydroxide salts) and as high as 50 (for formate salts), and may range from about 18 to 38 (e.g., for flouride and oxalate salts).

The concentration of promoter salt to be used depends on which embodiment of the invention is employed. When used as a means for nucleating water, concentrations are similar to those of the gaseous promoters, such as in the range of 100 to 150 ppm.

The R groups on the cations are, in certain embodiments, chosen so as to lower the solubility of trace gases, the incorporation of which into the CO₂ hydrate is undesirable. In certain embodiments, the R groups are chosen so that solubility of trace gases, the incorporation of which into the CO₂ hydrate is desirable, is increased. An example would be R groups with a mild chemical affinity for the solvated gas of interest, i.e., CO₂.

When used to alter the solubility of charged gases, the promoter structure away from the charged end is chosen to be chemically similar to the gaseous component whose solubility is to be decreased and chosen to have an affinity for gas molecules whose solubility is to be increased. Since alteration of gaseous solubility would typically be used in conjunction with the other embodiments (e.g. formation of mixed hydrates, raising of T, or lowering of P) the concentrations could be as high as about 30 wt. %, but in representative embodiments would be about 15 to about 20 wt. %.

In certain embodiments, the flue gas stream to be treated according to the subject methods is contacted with water that may contain CO₂ hydrate precursors or hydrate precursors of the promoter compounds. In this process, the nucleated water will include a CO₂ hydrate promoter, as described above. The CO₂ nucleated water employed in these embodiments of the subject invention comprises dissolved CO₂ in the form of CO₂ hydrate precursors, where the precursors are in metastable form. These precursors may be a composite of mixed hydrates containing both CO₂ and promoter molecules. The mole fraction of CO₂ in the CO₂ nucleated water ranges from about 0.01 to 0.10, such as from about 0.02 to 0.08, including from about 0.02 to 0.03. The temperature of the CO₂ nucleated water may range from about −5 to about 30° C., such as from about 7 to about 25° C., and including from about 10 to about 20° C. The temperature and pressure for formation of CO₂ hydrates may vary. The formation of CO₂ clathrates may occur under partial pressure conditions ranging from about 0.3 to about 40 atm, such as about 1 to about 30 atm and including from about 3 to about 20 atm.

The water that is used to produce the nucleated water may be obtained from any convenient source, where convenient sources include the deep ocean, deep fresh water aquifers, power-plant cooling ponds, and the like, and cooled to the required reactor conditions. In certain embodiments, nucleated water may be recycled from a downstream source, such as from one or more flash reactors (as described in greater detail below) where such recycled nucleated water may be supplemented as necessary with additional water, which water may or may not be newly synthesized nucleated water as described above and may, or may not, contain dissolved CO₂ hydrate promoters.

The amount of CO₂ that is dissolved in the water is determined in view of the desired CO₂ mole fraction of the CO₂ nucleated water to be contacted with the gaseous stream. One means of obtaining CO₂ nucleated water having relatively high mole fractions of CO₂ is to produce a slurry of CO₂ clathrates and then decompose the clathrates by lowering the pressure and/or raising the temperature of the slurry to release CO₂ and regenerate a partially nucleated water stream. Generally, nucleated water having higher mole fractions of CO₂ is desired because it more readily accepts CO₂ absorption or adsorption and accelerates the formation of other hydrate compounds. By high mole fraction of CO₂ is meant a mole fraction of about 0.02 to 0.04, such as from about 0.025 to 0.035.

The production of CO₂ nucleated water may conveniently be carried out in a nucleation reactor. The reactor may be packed with a variety of materials, where particular materials of interest are those which promote the formation of CO₂ nucleated water with hydrate precursors and include: stainless steel rings, carbon steel rings, metal oxides, and the like, to promote gas-liquid contact and enhance hydrate precursor formation. To ensure that the optimal temperature is maintained in the nucleation reactor, active coolant means may be employed. Any convenient coolant means may be used, where the coolant means may comprise a coolant medium housed in a container which contacts the reactor, preferably with a large surface area of contact, such as coils around and/or within the reactor or at least a portion thereof, such as the tail tube of the reactor. Coolant materials or media of interest include liquid ammonia, HCFCs, and the like. A particular coolant material of interest is ammonia, where the ammonia is evaporated at a temperature of from about −10 to about 10° C. The surface of the cooling coils, or a portion thereof, may be coated with a catalyst material, such as an oxide of aluminum, iron, chromium, titanium, and the like, to accelerate CO₂ hydrate precursor formation. Additionally, hydrate crystal seeding or a small (1-3 atm) pressure swing may be utilized to enhance hydrate precursor formation.

In certain embodiments of the subject invention, the CO₂ nucleated water is prepared by contacting water (e.g. fresh or salt water) with high pressure, substantially pure CO₂ gas provided from an external high pressure CO₂ gas source. In this embodiment, the water is contacted with substantially pure CO₂ gas that is at a pressure that is about equal to or slightly above the initial CO₂ partial pressure in the flue gas stream. As such, the pressure of the substantially pure CO₂ gas ranges in certain embodiments from about 5 to about 7 atm above the flue gas stream pressure (CO₂ overpressure stimulates hydrate precursor and hydrate formation). By substantially pure is meant that the CO₂ gas is at least 95% pure, such as at least 99% pure and including at least 99.9% pure. Advantages realized in this embodiment include the production of CO₂ saturated water that comprises high amounts of dissolved CO₂, e.g. amounts (mole fractions) ranging from about 0.005 to 0.025, such as from about 0.01 to 0.02. Additional advantages include the use of relatively smaller nucleation reactors (as compared to nucleation reactors employed in other embodiments of the subject invention) and the production of more CO₂ selective nucleated water. In those embodiments where small nucleation reactors are employed, it may be desirable to batch produce the CO₂ saturated water, e.g., by producing the total requisite amount of CO₂ saturated water in portions and storing the saturated water in a high pressure reservoir. The CO₂ saturated water is readily converted to nucleated water, i.e., water laden with CO₂ hydrate precursors, using any convenient means, e.g., by temperature cycling, contact with catalysts, pressure cycling, etc. This pre-structuring of the hydrate formation water not only increases the kinetics of hydrate formation, but also reduces the exothermic energy release in the CO₂ hydrate reactor. This, in turn, reduces the cooling demands of the process and increases overall process efficiency.

While the above protocols may be employed to prepare the initial nucleated water, in certain embodiments of interest, following the initial preparation of the nucleated water, additional nucleated water is obtained from the aqueous byproduct produced at the end of the process, such that recycled aqueous byproduct is employed as the nucleated water, as described in greater detail below.

As mentioned above, in the first step of the subject methods, the flue gas stream is contacted with an aqueous fluid, e.g., CO₂ nucleated water with or without hydrate promoters, under conditions of CO₂ hydrate formation. The aqueous fluid may be contacted with the gaseous stream using any convenient means. Preferred means of contacting the aqueous fluid with the gaseous stream are those means that provide for efficient removal, e.g., by absorption or adsorption which enhances hydrate formation, of the CO₂ from the gas through solvation of the gaseous CO₂ within the liquid phase or direct contact of the CO₂ gas with unfilled hydrate cages, which extract the CO₂ from the flue gas stream. Means that may be employed include concurrent contacting means, i.e., contact between unidirectionally flowing gaseous and liquid phase streams, countercurrent means, i.e., contact between oppositely flowing gaseous and liquid phase streams, and the like. Thus, contact may be accomplished through use of a fluidic Venturi reactor, sparger reactor, gas filter, spray, tray, or packed column reactors, and the like, as may be convenient.

Generally, contact between the flue gas stream and the aqueous fluid is carried out in a hydrate formation reactor. The reactor may be fabricated from a variety of materials, where particular materials of interest are those that catalyze the formation of CO₂ hydrates and include: stainless steel, carbon steel, and the like. The reactor surface, or a portion thereof, may be coated with a catalyst material, such as an oxide of aluminum, iron, chromium, titanium, and the like, to accelerate CO₂ hydrate formation. To ensure that the optimal temperature is maintained in the hydrate formation reactor, active coolant means may be employed. Any convenient coolant means may be used, where the coolant means may include a coolant medium housed in a container which contacts the reactor, such as the exit plenum and tail tube of the reactor, with a boiling aqueous phase. Coolant materials or media of interest include ammonia, HCFCs and the like. A particular coolant material of interest is ammonia, where the ammonia is maintained at a temperature of from about −10 to 10° C. Where the reactor includes gas injectors as the means for achieving contact to produce hydrates, the reactor may include 1 or a plurality of such injectors. In such reactors, the number of injectors will range from 1 to about 20 or more, where multiple injectors provide for greater throughput and thus greater hydrate production. Specific examples of various reactors that may be employed for hydrate production are provided in U.S. Pat. No. 6,090,186, the disclosure of which is herein incorporated by reference. In certain embodiments, the hydrate formation reactor is a finned tubular reactor, as described in greater detail below and in U.S. Pat. No. 6,797,039, the disclosure of which is herein incorporated by reference.

In certain embodiments, the hydrate formation reactor has a heat transfer surface area sufficient to transfer substantially all of said heat of formation energy produced by hydrate formation in said reactor to a coolant medium, e.g., such as those described above. By substantially all is meant at least about 95%, such as at least about 98%, including at least about 99% or more. In such embodiments, the hydrate formation reaction may be a convectively cooled tubular reactor, having a length to diameter ratio (L/D) that provides for the desired heat transfer surface area, where in representative embodiments the L/D ratio ranges from about 1000 to about 6000.

The hydrate formation conditions under which the gaseous and liquid phase streams are contacted, particularly the temperature and pressure, may vary. In certain embodiments of interest, the temperature at which the gaseous and liquid phases are contacted will range from about 30 to about 100° F., such as from about 32 to about 80° F., including from about 34 to about 60° F. The total pressure of the environment in which contact occurs, e.g., in the reactor in which contact occurs, may range from about 1 to about 25 atm, including from about 2 to about 20 atm, such as from about 3 to about 20 atm. The CO₂ partial pressure at which contact occurs generally does not exceed about 1.0 atm, and usually does not exceed about 0.5 atm. The minimum CO₂ partial pressure at which hydrates form in the presence of CO₂ hydrate promoters is generally less than about 1.0 atm, such as less than about 0.8 atm and may be as low as 0.5 or 0.4 atm or lower.

Upon contact of the CO₂—O₂ gaseous stream with the aqueous fluid, CO₂ is selectively removed from the gaseous stream by formation of CO₂ hydrates, which are formed as the CO₂ reacts with the CO₂ nucleated water liquid phase containing CO₂ hydrate precursors, with or without CO₂ hydrate promoters. Oxygen does not form hydrates, and remains as a gas.

In certain embodiments of the invention, CO₂ hydrate formation is conducted in a multi-stage process, employing two or more CO₂ hydrate reactors. In the first stage, the CO₂—O₂ gaseous stream is chilled and then compressed. Prior to chilling and compressing, any water vapor in the flue gas is condensed and the exiting gas stream is dried by applying heat. No further pretreatment or processing of the flue gas stream is required. In certain embodiments, a portion of the CO₂—O₂ is recycled to the combustor to control the flame temperature of the combustor, e.g., to reduce the flame temperature of the combustor. In certain embodiments, recycle rates may be 3 to 8 times the CO₂ production rate.

The compressed gas is then cooled and fed to the first stage CO₂ hydrate reactor. The products of the first stage CO₂ hydrate reactor are a hydrate slurry comprising 50-60 weight percent CO₂ hydrate and a CO₂—O₂ gaseous stream depleted in CO₂. Both of these products exit the first stage CO₂ hydrate reactor and are fed into a slurry/gas separator, wherein the CO₂-depleted flue gas and CO₂-rich slurry are separated.

The CO₂-depleted flue gas may be recompressed and chilled and fed into the second stage CO₂ hydrate reactor. Alternatively, the CO₂-depleted flue gas may be fed directly or compressed only a slight amount prior to entry into the second stage CO₂ hydrate reactor. If a CO₂ hydrate promoter is employed in the compression prior to the second stage CO₂ hydrate reactor may be employed.

The CO₂ hydrate promoter alters the thermodynamic conditions necessary to form CO₂ hydrates. The CO₂-depleted flue gas from the first stage reactor is again chilled and mixed with a chilled water stream, optionally containing a CO₂ hydrate promoter in the second stage CO₂ hydrate reactor. The products of the second stage CO₂ hydrate reactor are a hydrate slurry comprising 50-60 weight percent CO₂ hydrate and a CO₂—O₂ gaseous stream further depleted in CO₂. Both of these products exit the second stage CO₂ hydrate reactor and are fed into a second slurry/gas separator, wherein the further-depleted CO₂—O₂ gaseous stream and CO₂-rich slurry are separated.

At this point, the CO₂—O₂ gaseous stream may be vented into the atmosphere or recycled to the fuel combustor because about 85 to about 90% of the CO₂ has been extracted from the original CO₂—O₂ gaseous stream. Additional stages may be employed if further extraction of CO₂ is desired. The ammonia vapor or other working fluid produced in cooling the hydrate reactors may be used to regenerate CO₂ from the CO₂ hydrate slurries.

In certain embodiments of interest, in the first and second stages, the temperature to which the CO₂—O₂ gaseous stream is chilled will range from about 30 to about 60° F., such as from about 32 to about 55° F., including from about 34 to about 55° F. In the first and second stages, the thus-chilled CO₂—O₂ gaseous stream is compressed to a pressure of about 15 to about 25 atm, such as about 18 to about 23 atm, and including about 19 to about 21 atm. The temperature to which the compressed gas is cooled ranges, in certain embodiments, from about 30 to about 60° F., such as from about 32 to about 55° F., and including from about 34 to about 55° F. The CO₂ concentration in the CO₂—O₂ gaseous stream exiting the first stage CO₂ hydrate reactor ranges, in certain embodiments, from about 70 to about 80 weight percent, such as from about 70 to about 75 weight percent, and including from about 72 to about 74 weight percent. The CO₂ concentration in the CO₂—O₂ gaseous stream exiting the second stage CO₂ hydrate reactor will, in certain embodiments, have been reduced to 50 weight percent or less.

Any convenient gas-liquid phase separation means may be employed, where a number of such means are known in the art. In representative embodiments, the gas-liquid separator that is employed may be a vertical or horizontal separator with one or more, such as a plurality of, gas off-takes on the top of the separator. The subject invention provides for extremely high recovery rates of the multi-component gaseous stream. In other words, the amount of oxygen and trace gases removed from the flue gas stream following selective CO₂ extraction according to the subject invention is extremely low. For example, where the flue gas stream is a power-plant flue gas stream, the amount of gases (i.e., O₂) recovered is above 85.0%, in certain embodiments above 90.0% and in certain embodiments above 95.0%, where the amount recovered ranges in certain embodiments from about 85.0 to 99.0%.

Separation of the slurry and gaseous products of the hydrate formation reactors produces separate slurry and gaseous product streams, each at low pressure, where by low pressure is meant a pressure ranging from about 3 to about 20 atm, such as from about 2 to about 15 atm. In certain embodiments, compression energy is then recovered from the gaseous product stream. Compression energy may be recovered from the gaseous product using any convenient protocol, such as by passing the gas through a gas expansion turbine. Such embodiments provide significant benefits with respect to reducing overall net compression energy requirements of the process while permitting higher working fluid compression ratios, which in turn provide for more efficient CO₂ separation.

In certain embodiments, the product CO₂ hydrate slurries are then compressed using a liquid or slurry reciprocating pump (as opposed to a gas compressor) to raise the pressure of the CO₂ hydrate slurries from a first to a second pressure (that is higher than the first pressure) and to produce a high-pressure CO₂ hydrate product. The first pressure, as indicated above, ranges in certain embodiments from about 3 to about 20 atm, such as from about 5 to about 15 atm. As reviewed above, the high-pressure CO₂ product is a product having a pressure that is at least about 3 fold higher, and sometimes at least about 5-fold higher or at least about 10-fold higher, or even higher than the pressure at which the CO₂ separation from the gaseous stream occurred. In certain embodiments, the second pressure ranges from about 50 to about 70 atm, such as from about 55 to about 65 atm, and including from about 60 to about 63 atm. Any convenient liquid or slurry pump may be employed in this step of the subject methods, where the pump may be made of one or a plurality, e.g., two or more, individual pumps or pump elements, e.g., slurry pumps, etc.

Where desired, high-pressure CO₂ gas can easily be regenerated from the CO₂ hydrates, e.g., where high pressure CO₂ gas is to be a product or further processed for sequestration. The resultant CO₂ gas may be disposed of by transport to the deep ocean or ground aquifers, or used in a variety of processes, e.g., enhanced oil or gas recovery, coal bed methane recovery, or further processed to form metal carbonates, e.g., MgCO₃, for fixation and sequestration.

In certain embodiments, the CO₂ hydrate slurries are treated in a manner sufficient to decompose the hydrate slurries into high pressure CO₂ gas and a high pressure mixed promoter/CO₂ nucleated water stream, i.e., it is subjected to a decomposition step. In certain embodiments, the CO₂ hydrate slurries are thermally treated, e.g., flashed in a flash reactor/regenerator, where by thermally treated is meant that temperature of the CO₂ hydrate slurry is raised in sufficient magnitude to decompose the hydrates and produce CO₂ gas. Each hydrate reactor will have a corresponding thermal treatment means. In certain embodiments, the temperature of the CO₂ hydrate slurry is raised to a temperature of between about 65 to 105° F., at a pressure ranging from about 40 to about 60 atm. One convenient means of thermally treating the CO₂ hydrate slurries is in counterflow heat exchangers, where each heat exchanger comprises a heating medium in a containment means that provides for optimal surface area contact with the hydrate slurry. Any convenient heating medium may be employed, where specific heating media of interest include: ammonia, HCFCs and the like, with ammonia vapor at a temperature ranging from 20 to 40° C. being of particular interest. In certain embodiments, the ammonia vapor is that vapor produced in cooling the nucleation and/or hydrate formation reactors, as described in greater detail in terms of the figure.

Flash regenerated CO₂ will have a pressure of about 40 to about 50 atm as it exits the flash reactor. Where desired, the pressure of the CO₂ product gas may be increased to a third pressure, e.g., ranging from about 100 to about 150 atm, using any convenient means, e.g., a gas compressor, for sequestration or for subsequent use as described above. Because the CO₂ is regenerated at high pressures from the flash reactors, the power requirements for the final compression are reduced greatly.

The high-pressure water streams produced in each flash reactor may be chilled and recycled to the respective hydrate reactors. Make-up water and/or promoter may be added as desired. The recycle stream to the second (or subsequent) stage reactor may or may not contain a promoter compound, depending upon whether a promoter was employed in the second stage hydrate reactor. In certain embodiments, compression energy is recovered from this high-pressure aqueous byproduct, e.g., by use of a pressure recovery turbine.

Multi-component gaseous streams (containing CO₂—O₂) that may be treated according to the subject methods include oxidizing condition streams, e.g., flue gases from combustion utilizing oxygen. Particular multi-component gaseous streams of interest that may be treated according to the subject invention include oxygen containing combustion power plant flue gas, and the like.

Where desired, the above process may be modified further to include use of a gaseous hydrate promoter, e.g., as described in U.S. Pat. No. 6,352,576 and U.S. Pat. No. 6,797,039, the disclosures of which are herein incorporated by reference. In these embodiments, a CO₂—O₂ stream that includes an amount of a gaseous CO₂ hydrate promoter is subjected to hydrate formation. The amount of CO₂ hydrate promoter that is present in the CO₂—O₂ stream may be sufficient to provide for a reduction in the CO₂ partial pressure requirement of hydrate formation, as described in U.S. Pat. No. 6,352,576, the disclosure of which is herein incorporated by reference.

The specific amount of gaseous CO₂ hydrate promoter that is present in the provided CO₂—O₂ stream depends, in large part, on the nature of the CO₂—O₂ stream, the nature of the CO₂ hydrate promoter, and the like. In certain embodiments, the amount of gaseous CO₂ hydrate promoter that is present, initially, in the CO₂—O₂ stream ranges from about 1 to 5 mole percent, such as from about 1.5 to 4 mole percent, and including from about 2 to 3 mole percent, in many embodiments.

Any convenient gaseous CO₂ hydrate promoter that is capable of providing the above-described reduction in CO₂ partial pressure requirement of hydrate formation when present in the CO₂—O₂ stream may be employed.

One type of gaseous CO₂ hydrate promoter is a sulfur containing compound, where specific sulfur containing compounds of interest include: SO₂, CS₂, and the like. Where the CO₂ hydrate promoter is SO₂, it is generally present in the CO₂—O₂ stream in an amount ranging from about 0.3 to about 2.0 mole percent, such as from about 0.5 to about 1.5 mole percent, and including from about 0.7 to about 1.1 mole percent.

In certain embodiments, a CO₂—O₂ gaseous stream of interest will be tested to ensure that it includes the requisite amount of CO₂ hydrate promoter of interest. In certain embodiments where desired, a sufficient amount of the CO₂ hydrate promoter is added to the CO₂—O₂ gaseous stream to be treated. The requisite amount of CO₂ hydrate promoter that needs to be added to a given CO₂—O₂ gaseous stream of interest necessarily varies depending on the nature of the gaseous stream, the nature of the CO₂ hydrate promoter, the desired CO₂ separation ratio and the like. The requisite amount of CO₂ hydrate promoter may be added to the CO₂—O₂ gaseous stream using any convenient protocol, e.g., by combining gaseous streams, adding appropriate gaseous components, etc.

Systems

As summarized above, also provided are systems for use in practicing the subject methods. A feature of the subject systems is that they include at least: (a) two or more hydrate formation reactors; and (b) two or more slurry compression elements for pumping CO₂ hydrate slurries produced by the hydrate formation reactors.

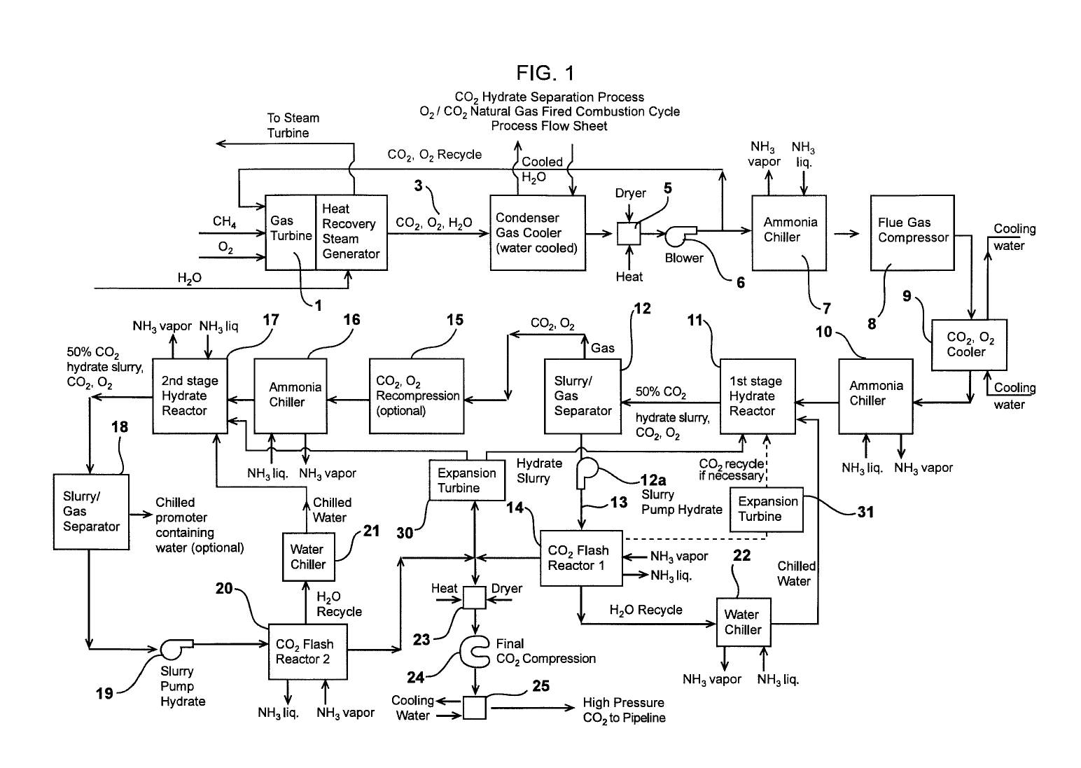

The invention will now be further described in terms of representative embodiments of the subject systems. One representative embodiment of the subject systems is shown schematically in FIG. 1. FIG. 1 provides a schematic flow diagram of a system 100 for selectively removing CO₂ from a CO₂—O₂ gaseous stream in a manner according to the present invention. In FIG. 1, natural gas (CH₄) is combusted with oxygen of 95% or greater purity in a gas turbine 1, followed by a heat recovery steam generator 2 in a gas turbine-steam turbine combined cycle power plant. The combustion products 3, primarily water vapor, CO₂, excess O₂, and trace gases (e.g., argon, nitrogen oxides, etc.), has a temperature ranging from about 130 to about 180° F., e.g., 150° F., and a pressure of about 1 atm. The water vapor is condensed in a water-cooled condenser/gas cooler 4. The exiting gas stream, made up of O₂ (5-15%) and CO₂ (85-95%), is dried in a thermal dryer 5 and blown by blower 6 into an ammonia chiller 7. The ammonia chiller is cooled with liquid ammonia. The ammonia vapor produced is used subsequently in the flash reactors. The chilled flue gas is then compressed in compressor 8, cooled in a water-cooled cooler 9, and then chilled in an additional ammonia chiller 10. The chilled flue gas is passed into the first stage CO₂ hydrate reactor 11, which is cooled by liquid ammonia and chilled water. The product, which is 50 wt. % CO₂ hydrate slurry and 50 mole % unreacted CO₂—O₂, is passed through a slurry/gas separator 12. The separated CO₂ hydrate slurry is pumped to high pressure with slurry pump 13 and sent to the first CO₂ flash reactor 14. Ammonia vapor is input into the flash reactor 14, and the liquid ammonia by-product is recycled to the ammonia chillers. The CO₂—O₂ from the slurry/gas separator 12 is recompressed in compressor 15, chilled in ammonia chiller 16, and sent to the second stage CO₂ hydrate reactor 17, which is cooled by liquid ammonia and chilled water. The product, which is 50 wt. % CO₂ hydrate slurry and 50 mole % unreacted CO₂—O₂, is passed through a second slurry/gas separator 18. The separated CO₂ hydrate slurry is pumped to high pressure with slurry pump 19 and sent to the second CO₂ flash reactor 20. Ammonia vapor is input into the flash reactor 20, and the liquid ammonia by-product is recycled to the ammonia chillers. The water produced in flash reactors 14 and 20 is recycled into the CO₂ hydrate reactors 11 and 17 after being cooled in water chillers 21 and 22. The regenerated CO₂ from flash reactors 14 and 20 is dried in thermal dryer 23 and compressed to high pressure in final compressor 24. The high pressure CO₂ is cooled in water cooler 25, and the product high pressure CO₂ is sent to a pipeline for subsequent use or sequestration. A small portion (3-5%) of the produced CO₂ may be chilled and recycled, if necessary, to resaturate with CO₂ the chilled water streams entering the first and/or second CO₂ hydrate reactors 11 and 17.

The subject methods and systems provide for the resource efficient regeneration of high pressure CO₂ from an initially low pressure CO₂ hydrate reactor and slurry/gas separator. The subject methods and systems provide for numerous opportunities to reduce parasitic energy loss, and efficiently provide for separation of CO₂ from a flue gas stream to produce a high pressure CO₂ product gas. As such, the subject invention represents a significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for removing CO₂ from an oxygen combustion gaseous stream said method comprising:
(a) contacting an oxygen combustion gaseous stream with an aqueous fluid in a first hydrate reactor under conditions sufficient to produce a CO₂ hydrate slurry and a CO₂ depleted gaseous stream; and
(b) separating said CO₂ depleted gaseous stream from said CO₂ hydrate slurry;
wherein the CO₂ depleted gaseous stream from the separating step (b) is sent to a second hydrate reactor.

2. The method of claim 1, which further comprises compressing said CO₂ hydrate slurry from a first pressure to a second pressure that is higher than said first pressure to produce a high pressure CO₂ hydrate slurry product.

3. The method of claim 1, which further comprises decomposing high pressure CO₂ hydrate slurry to produce a high-pressure CO₂ product.

4. The method according to claim 1, which is conducted in at least two stages.

5. The method according to claim 1, wherein said CO₂ hydrate slurry and said CO₂ depleted gaseous stream are separated at low pressure.

6. The method according to claim 5, wherein said first pressure ranges from about 3 to about 20 atm.

7. The method according to claim 1, wherein said compressing is performed with a liquid or slurry reciprocating pump.

8. The method according to claim 1, wherein said second pressure ranges from about 40 to about 60 atm.

9. The method according to claim 1, wherein said decomposing is performed in a flash reactor (regenerator).

10. The method according to claim 9, wherein the high-pressure CO₂ product has a pressure ranging from about 40 to about 50 atm as the CO₂ product exits the flash regenerator.

11. The method of claim 1, wherein said aqueous fluid of said contacting step is CO₂ nucleated water.

12. The method according to claim 11, wherein said aqueous fluid of said contacting step comprises a CO₂ hydrate promoter.

13. The method according to claim 12, wherein said CO₂ hydrate promoter is a low molecular weight compound.

14. The method according to claim 13, wherein said low molecular weight compound is an organic salt.

15. The method according to claim 14, wherein said organic salt is an alkyl-onium salt.

16. The method according to claim 1, wherein said contacting step occurs in a reactor having a heat transfer surface area sufficient to transfer substantially all of said heat of formation energy produced by hydrate formation in said reactor to a coolant medium.

17. The method according to claim 9, wherein said reactor has a length to diameter ratio (L/D) that ranges from about 100 to about 6000.

18. The method according to claim 1, wherein said separating step (b) occurs in a low-pressure liquid/gas separator.

19. The method according to claim 18, wherein said method further comprises recovering compression energy from said CO₂ depleted gaseous stream produced by said separating step (b).

20. The method according to claim 1, wherein said method further comprises reducing the temperature and increasing the pressure of said oxygen combustion flue gas stream prior to said contacting step (a).

21. The method according to claim 1, wherein said method further comprises producing CO₂ gas from said high-pressure CO₂ hydrate slurry product.

22. The method according to claim 21, wherein said CO₂ gas is produced from said high-pressure CO₂ hydrate slurry product by flashing said high pressure CO₂ hydrate slurry product.

23. The method according to claim 21, wherein said method further comprises compressing said CO₂ gas to a third pressure that is higher than said second pressure.

24. The method according to claim 23, wherein said third pressure ranges from about 100 to about 150 atm.

25. The method according to claim 21, wherein said CO₂ gas producing step also produces an aqueous byproduct that is recycled for use in further CO₂ hydrate formation.

26. The method according to claim 25, wherein said method comprises recovering compression energy from said aqueous byproduct.

27. A system for selectively removing CO₂ from an oxygen combustion flue gas stream to produce a CO₂ depleted gaseous stream, said system comprising:
(a) at least two stages of hydrate formation reactors; and
(b) at least two stages of slurry pump elements for compressing the CO₂ hydrate slurries produced by said hydrate formation reactors.

28.-37. (canceled)
